SEQUENCE LISTING (3 sequences)

SEQ ID NO: 1 - DNA, 1819 bp, Streptomyces coelicolor
ggatccgtccgtcccatggcctcgaaccccagcgtcgtcgccgcgatcaccgccagcgcgtcgcccgtggcgacgtcgtccgccagggcgccgtccagggcgaccacggtcaggccgctgctcacccagtccacccagtggcggcgcagatcggcccgttccttccaggtcgtgtccccgctcagcccgaaaccggcccgcagcacgacatcgtcctggagtcgctgggccagcgtgtgcgaggtgtcgacgagggactggagcgagggcgtggccccctccggccacgcgtgggcgatctgccggagcaccgacaacgcctcgccctgcaccgcctcggccaccgcgttcttgttcgcgaagtggaagtgcagtgccccgttgctcaccccggcccgggagctgatcatcgtgagcgacgcgggcgcgaacccctcctggtcgaacacctcggcggcggagagaatcagagctcgacgcgtacgtgccgcccgctcctgcttggtcatgtcgtgctccgtggtcgcgatcgtcgagccccaaggcgggataagtggcaccccagtgtcacgtgccgcccccaccgggcagtaatcgcgcgaccaccacgcgctccctctggcggatctcatgctgtccaccagcggcagggtccaccctggtcccgtccgctccggtcctgacccgcaccggattcgggttcgccgcgggcgggccgcactcgtcgaacggtaaggggaaaaggcgatgggaagtgactggttcaggcggttcggtcccgcacccgacagcgacgtccggctgatctgcctcccgcacgcgggcggcgccgcgggagcgttcctcgcgctggcgcgggagctgacgcccgagttcgacgtcctgtccgtgcagtacccgggccgccaggaccgcaggcgggaaccgccgctcgcggacatcggcctgctggtggacgcgctcgccggggagatggccccgctcgccgaccgcccccacgcgttcttcgggcacagcatgggcgcgctgctcgcctacgaactcgcccgcgaactgcggcggcgcgccctgcccggaccgtgccacctgttcctgtccggccggttcgcgcccacgccgcagggcagtgacagcgaccggctggacaccgacgagaaggtcatcgccatgatccgccggctcggcggcacggtggggaaggtcttcgacgacccggacgtgatggagatggtgatgccgccgctgcgcgccgactaccgcgcggtcggggcctacacctggcagccgggtccgccgctcgccgtcccggtcaccgtcctggtcggggaccaggaccccgtcgttcccgtggcggcggccgccgcgtggcgcgagcacaccaccgccggatccgacctgagggtgttgccgggcggtcacttctatctcgaccagagcgtgccggaggtcgccggtatcgtccggtccgcactgcgcgccacggccgtacccggccccgggtggcgggcgttgcttcatggtgagtccccagtgggtactcctggcccacaatgaaacggaccgctcggtctcttctctcgactccgcggtcccgccacccggcggccggtgagtcccgcgttccgcggcggccggatctccgtgtacgtacgacatccgcggcacggcaaaccgtgtgccgggacaggagtgtgaggcagtgcggttcaatctcatgggcccgttcgagatcgtcggcgacgacggcagggtgcacctgcccaccacgcccaaggtctgccagacactcgccctgctgctgacccggccgaacgagatc

SEQ ID NO: 2 - protein, 268 aa, Streptomyces coelicolor
MGSDWFRRFGPAPDSDVRLICLPHAGGAAGAFLALARELTPEFDVLSVQYPGRQDRRREPPLADIGLLVDALAGEMAPLADRPHAFFGHSMGALLAYELARELRRRALPGPCHLFLSGRFAPTPQGSDSDRLDTDEKVIAMIRRLGGTVGKVFDDPDVMEMVMPPLRADYRAVGAYTWQPGPPLAVPVTVLVGDQDPVVPVAAAAAWREHTTAGSDLRVLPGGHFYLDQSVPEVAGIVRSALRATAVPGPGWRALLHGESPVGTPGPQ

SEQ ID NO: 3 - DNA, 826 bp, Streptomyces coelicolor
atgggaagtgactggttcaggcggttcggtcccgcacccgacagcgacgtccggctgatctgcctcccgcacgcgggcggcgccgcgggagcgttcctcgcgctggcgcgggagctgacgcccgagttcgacgtcctgtccgtgcagtacccgggccgccaggaccgcaggcgggaaccgccgctcgcggacatcggcctgctggtggacgcgctcgccggggagatggccccgctcgccgaccgcccccacgcgttcttcgggcacagcatgggcgcgctgctcgcctacgaactcgcccgcgaactgcggcggcgcgccctgcccggaccgtgccacctgttcctgtccggccggttcgcgcccacgccgcagggcagtgacagcgaccggctggacaccgacgagaaggtcatcgccatgatccgccggctcggcggcacggtggggaaggtcttcgacgacccggacgtgatggagatggtgatgccgccgctgcgcgccgactaccgcgcggtcggggcctacacctggcagccgggtccgccgctcgccgtcccggtcaccgtcctggtcggggaccaggaccccgtcgttcccgtggcggcggccgccgcgtggcgcgagcacaccaccgccggatccgacctgagggtgttgccgggcggtcacttctatctcgaccagagcgtgccggaggtcgccggtatcgtccggtccgcactgcgcgccacggccgtacccggccccgggtggcgggcgttgcttcatggtgagtccccagtgggtactcctggcccacaatgagacccggccgaacgagatc

(19) United States
(12) Patent Application Publication
(10) Pub. No.: US 2005/0153413 A1
(43) Pub. Date: Jul. 14, 2005

(54) Type II thioesterase from streptomyces coelicolor a3(2) and the coding sequence

(76) Inventors: Katarzyna Kuczek, Wroclaw (PL); Barbara Kuczek, Wroclaw (PL); Magdalena Kotowska, Wroclaw (PL); Krzysztof Pawlik, Wroclaw (PL); Daniela Wiernik, Wroclaw (PL)

Correspondence Address: CLARK & ELBING LLP, 101 FEDERAL STREET, BOSTON, MA, 02110, US

(21) Appl. No.: 10/473,092

(22) PCT Filed: Mar. 28, 2002
(86) PCT No.: PCT/PL02/00027

(30) Foreign Application Priority Data
Mar. 28, 2001 (PL) P-346786

(51) Int. Cl. C12P 19/56; C07H 21/04; C12N 9/10; C12N 1/21; C12N 15/74

(52) U.S. Cl. 435/193; 435/78; 435/69.1; 435/320.1; 435/252.3; 536/23.2

(57) ABSTRACT

The invention concerns a biosynthesis of nonaramatic polyketide compounds. More particularly, the present invention relates to a isolated polynucleotide molecule, a nucleic acid vector comprising the molecule, and isolated polypeptide and uses thereof. A new, type II thioestrerase can be obtained as an application outcome of this invention. The enzyme is active in polyketide, especially macrolide, biosynthesis when associated with a multienzyme complex of a polyketide synthase. The activity of the enzyme results in a increase of polyketide biosynthesis efficiency.

The present invention relates generally to the fields of molecular biology. More particularly, it concerns a biosynthesis of nonaromatic polyketide compounds.

More particularly, the present invention relates to a isolated polynucleotide molecule, a nucleic acid vector comprising the molecule, an isolated polypeptide and uses thereof. A new, type II thioestrerase can be obtained as an application outcome of this invention. The enzyme is active in polyketide, especially macrolide, biosynthesis when associated with a multienzyme complex of a polyketide synthase. The activity of the enzyme results in an increase of polyketide biosynthesis efficiency.

Polyketides are a large and structurally a diverse group of compounds synthesized by microorganisms (bacteria and fungi) and by plants. The compounds are synthesized as a result of multiple, small carboxylic acid condensation and reduction cycles. Most of the known, naturally occurring polyketides, are produced by soil bacteria, streptomycetes. Polyketides are known as biologically active compounds, most of which are commonly used antibiotics, immunomodulators and anticancer drugs.

Nascent polyketides are processed by large multienzyme complexes, polyketide synthases (PKS). In the type I PKS, involved in production of macrolide antibiotics such as erythromycin, reactions of the biosynthetic cycle are catalysed sequentially by separate enzymic domains housed in large multifunctional polypeptides. Each complete cycle of condensation and reduction reactions is catalysed by a module, a functional unit of the PKS. The substrate acyl chains which undergo successive reactions are tethered as thioesters by acyl carrier domains of the PKS polypeptides. A terminal thioesterase domain (TE) catalyses release and cyclization of the full-length (fully processed) polyketide chain (Katz & Donadio, 1993).

Many type I PKS, and also nonribosomal peptide synthetase (NRPS) clusters, contain additional TE genes located adjacent to the PKS genes within the cluster of antibiotic biosynthetic genes (Weissman et al., 1998; Schneider & Marahiel, 1998; Shaw-Reid et al., 1999; August et al., 1998; Xue et a!., 2000; Heathcote et al., 2001). The products of such genes are discrete proteins called type II thioesterases (TE II) to distinguish them from chain-terminating thioesterase domains, TE I (Gokhale et al., 1999).

The function of type II thioesterases is predicted from gene disruption analysis, complementation studies, and determination of their substrate specificities (Weissman et al., 1998; Butler et al., 1999; Heathcote et al., 2001). Polyketide production is drastically reduced, by 90% or more, in strains with deleted TE II gene (Xue et al., 2000; Butler et al., 1999; Doi-Katayama et al., 2000), indicating an important function, proposed to involve editing of aberrant intermediates (Butler et al., 1999) during the course of polyketide biosynthesis. More recently, this hypothesis has been confirmed and the mechanism clarified. Thus, the TylO protein displayed hydrolytic activity in vitro towards short chain acyl-CoAs, indicating that the enzyme could remove aberrantly decarboxylated (and, therefore, non-reactive) extender acyl chains from the PKS during polyketide biosynthesis (Heathcote et al., 2001). By hydrolytic release of such aberrant acyl groups, TE II was proposed to unblock PKS modules and restore overall efficiency of the complex enzyme.

Modular polyketide synthases, as demonstrated by numerous experiments, can serve as efficient tools for the combinatorial biosynthesis of new polyketides. The latter include both analogues of existing polyketides and compounds with a novel activity. Genetic engineering studies allow assembly of novel polyketide chains following fusion, swapping or repositioning of catalytic domains, modules or whole peptides within PKS polypeptides (Hutchinson & Fujii, 1995; Ranganathan et al., 1999; Tang et al., 1999). In engineered PKSs, co-expression of TE IIs in addition to other PKS proteins, might help in achieving elevated levels of the polyketide products.

This invention is directed towards production of a new, type II thioesterase as an expression product of the new gene. The activity of the enzyme might play a beneficial role in nonaromatic polyketide synthesis.

The subject of this invention is an isolated polynucleotide comprising the sequence having at least 60% homology to the nucleic acid sequence of SEQ ID NO. 1 or its complementary strand or fragments thereof. Preferably, the polynucleotide comprises the sequence having at least 70% homology to the nucleic acid sequence of SEQ ID NO:1 or its complementary strand or fragments thereof encoding TE II protein. More preferably, the polynucleotide comprises the sequence having at least 80%, and more preferably at least 90%, homology to the nucleic acid sequence of SEQ ID NO:1 or its complementary strand or fragments thereof. Preferably the polynucleotide encodes tioesterase type II proteine. Preferably the polynucleotide according to the invention encodes at least 15, and more preferably at least 150, contiguous amino acids from SEQ ID NO:2. Preferably, the polynucleotide encodes polypeptide comprising the amino acid sequence of SEQ ID NO:2. Preferably the polynucleotide comprises the sequence of SEQ ID NO:1 or SEQ ID NO:3 or complementary strand thereof.

In accordance to the invention, the DNA can be used for the expression of TE II, both as a coding and a regulatory element. The DNA can be used to obtain antisense strand and to design hybridization probes and primers.

The other subject of the invention is any nucleic acid vector comprising the polynucleotide according to the invention, as defined above. The vector can contain e.g. the entire sequence SEQ ID No. 1, a functional equivalent or a specified element thereof, like the sequence encoding TE II protein, a fragment of the protein or the regulatory element.

The subject of the invention is also a protein or fragment thereof encoded by a polynucleotide according according to the invention, as defined above.

The subject of the invention is also an application of a polynucleotide according to invention for the expression of a protein, preferably the protein of TE II activity.

Applications of a polypeptide of an amino acid sequence essentially consistent with SEQ ID No. 2 or fragments thereof for polyketide synthesis are also addressed by the invention. The polypeptide can be used to obtain multienzyme polyketide synthases and/or to increase biosynthetic efficiency of polyketide synthase complexes.

By the use of the methods similar to the ones disclosed in the examples given below, the TE II gene from the chromosome of Streptomyces coelicolor A3(2) disclosed in this invention can be transferred to the chromosome of another streptomycete strain which produces a polyketide (possibly an antibiotic). Based on the disclosed results, one can expect that the transferred gene will be expressed and a functional protein of the TE II activity will be produced. This would increase the efficiency of the polyketide production. Thus the protein can be used for polyketide synthesis. Especially, the protein can be used to obtain new multienzyme polyketide synthases and to increase a biosynthesis performed by known polyketide synthase complexes. A relatively broad substrate tolerance exhibited by the enzyme would help to apply the TE II in different, heterologous synthase complexes.

### EXAMPLE 1

### Isolation and Structure of the Gene scoT, Encoding a New TE II

Hybridization experiment was done by using DNA isolated from cosmid clones from the geneomic DNA library of the streptomycete—Streptomyces coelicolor A3(2) M145. Nonradioactively-labeled DNA, obtained as a PCR reaction product, was used as a probe. The substrate for nonradioactive labeling in a course of random priming reaction (Sambrook et al., 1989) was digoxygenin-11-dUTP. Probe labeling, hybridization and hybrid detection was done according the procedure of Boehringer Mannheim (The DIG System User's Guide for Filter Hybridization). The amino acid sequence of the probe was equivalent to the C-terminal region of ketosynthase domains from 6-deoxyerythronolide B synthase of Saccharopolyspora erythraea (synthesizing an aglycone part of erythromycin A). Based on a hybridization with the probe, a cosmid clone was chosen for further studies. In a course of these studies the restriction map of the cosmid was obtained. Another DNA probe of a sequence comprising a central part of acyltransferase domain from type I polyketide synthase, including active site region, was used. Similar to the former probe, the further one was a digoxygenin-labeled, PCR reaction product. The DNA reacting with the further probe, obtained as two restriction fragments, was cloned in pBlueScript SK vector (Stratagene) in E. coli XL1Blue strain (Stratagene) by using the procedures of Sambrook et al., (1989). The fragments were further cloned into the same vector as a one, continuous insert.

DNA sequence of the cloned restriction fragments was obtained. The sequence of S. coelicolor DNA was determined both manually (Kuczek et al., 1998) by using the chain-termination method with the Sequenase v. 2.0 sequencing kit of Amersham and by automated sequencing performed by Qiagen Sequencing Services, Hilden, Germany. The sequence was determined on both strands and submitted to the GenBank database (AF109727). The sequence AF109727, originally deposited in the database, contained numerous frame-shift mistakes which were later corrected and the corrected sequence (SEQ ID No.1) was analysed. Computer-assisted sequence analysis, with the aid of CODONPREFERENCE program, revealed an open reading frame of 807 bp, located on the two fragments. The ORF designated scoT (SEQ ID No.3) was deduced to code for a protein of 268 amino acid residues (molecular mass 28 686 Da, isoelectric pH 6.17) of which about 53% were predicted to be hydrophobic (SEQ ID No.2).

Comparison of the scoT sequence with others in the databases revealed extensive similarities with thiosterase enzymes from various actinomycetes and other bacteria and also rat S-acyl fatty acid synthase complex. The greatest similarity was found with type II thioesterases (Pfam00975), namely, 43% identity with a TE II (AF040570) from the rifamycin biosynthetic gene cluster of Amycolatopsis mediterranei, 43% identity with a TE II (X60379) associated with DEBS (6-deoxyerythronolide B synthase) from Saccharopolyspora erythraea, and 40% identity with TylO (U08223), the TE II involved in tylosin biosynthesis in Streptomyces fradiae. ScoT (SEQ ID No.2) is predicted to belong to the well-known alpha/beta hydrolase family (Pfam00975). Comparisons of the nucleotide and amino acid sequences with the databases were performed with the BLAST and ClustalW programs.

Comparison of the SEQ ID No.1 sequence with others in the databases also revealed that the sequence comprises a region active in a transcriptional regulation of scoT.

### EXAMPLE 2

### Expression of scoT and Confirmation of ScoT Protein Activity: Complementation of a Knockout Mutant of the S. fradiae Natural TE II Gene by the TE II Gene of S. coelicolor A3(2)

We attempted to determine whether the product of scoT has TE II activity. This was examined by studying whether it could functionally replace the native TE II (i.e. TylO) in the tylosin producer, Streptomyces fradiae. ScoT and TylO show extensive amino acid sequence similarity and the complementation system for TE II gene-disrupted S. fradiae strain was constructed (Butler et al., 1999). Therefore, scoT was cloned in a conjugative expression vector pLST9828 and integrated upon transconjugation from E. coli into the chromosome of a tylO-disrupted strain of S. fradiae. The vector was constructed in the laboratory of Prof. E. Cundliffe (University of Leicester, Leicester, U.K.).

There are two unique restriction sites suitable for cloning in pLST9828: BamHI and XbaI. As BamHI site is also present within the scoT sequence, the gene was ligated into pLST9828 in a two-step process. First, the DNA containing the C-terminal part of ScoT was PCR amplified from the pBSK(−) plasmid clone template using M13 reverse primer and the TE-Rev primer with an engineered XbaI site (underlined): 5′-TTTTCTAGATGTCGTACGTACACGGA-3′. The PCR product was purified using the Qiaex II DNA purification kit, digested and ligated into pLST9828 using the BamHI and xbaI sites. Then, a second PCR product containing the N-terminal part of ScoT was obtained from the template of another pBSK(+) construct using the T7 universal primer and the TE-Fw primer with an engineered BanHI site (underlined): 5′-TTTTTTGGATCCGATGGGAAGTGACTGGTT-3′. The 50 μl reaction mixture contained 5 μl of 10×PCR DyNAzyme buffer (Finnzymes), 1 μl of 10 mM deoxynucleoside triphosphate mixture, 50 pmol of each oligonucleotide, about 10 ng of template DNA and 1 μl of DyNAzyme™ II DNA polymerase (Finnzymes). Cycling was as follows: hot start at 96° C. for 6 min, 1 min at 80° C. (adding of the enzyme), 31 cycles with denaturation at 95° C. for 1 min, annealing at 63-65° C. for 1 min and extension at 72° C. for 1.5 min, followed by a final extension at 72° C. for 5 min. The product was digested with BamHI and ligated into the pLST9828 derivative obtained in the first step of the cloning procedure. Gentamycin (15 μg ml⁻¹) was used for selection of E. coli DH5α transformants. The authenticity and orientation of the cloned fragments was confirmed by automated sequence analysis.

The gene cloned in pLST9828 was introduced, by transconjugation from E. coli S17-1 (Kieser et al., 2000), into S. fradiae strain, a disruption mutant of the native TE II gene, tylO. Due to a polar effect of the disruption of tylO on the expression of the downstream gene, the tylO-disrupted strain produce demycarosyl-tylosin (desmycosin) as do strains in which the disruption is successfully complemented by cloned DNA (Butler et al., 1999).

Complemented mutant strains of S. fradiae were fermented and fermentation products were extracted and analysed by reverse phase HPLC, with absorbance measurement at 282 nm (Butler et al., 1999). Desmycosin was used as an internal standard to identify fermentation products in complemented strains. Transconjugation, as well as fermentation and product analyses were done in the laboratory of Prof. E. Cundliffe.

Desmycosin production was restored up to 48% of the level of macrolide produced by the wild type strain. Control fermentation of the non-complemented, tylO-disrupted strain yielded only minimal amounts of desmycosin. These results showed that the TE II gene, scoT, from Streptomyces coelicolor A3(2) could, by complementation, restore to a significant level macrolide production in the tylO-disrupted strain of Streptomyces fradiae. The two enzymes appear, therefore, to be equivalent in their catalytic function.

Literature

August, P. R., Tang, L., Yoon, Y. J., Ning, S., Muller, R., Yu, T. W., Taylor, M., Hoffmann, D., Kim, C. G., Zhang, X., Hutchinson, C. R. & Floss, H. G. (1998) Chem. Biol., 5, 69-79.
Butler A. R., Bate N., Cundliffe E. (1999) Chem. Biol. 6: 287-292
Doi-Katayama Y., Yoon Y. J., Choi Ch. Y., Yu T. W., Floss H. G., Hutchinson C. R. (2000) J. Antibiotics 53, 484-495.
Gokhale R. S., Hunziker D., Cane D. E., Khosla Ch. (1999) J. Chem. Biol. 6: 117-125
Heathcote M. L., Staunton J., Leadlay P. F. (2001) Chem. Biol. 8: 207-220
Hutchinson C. R., Fujii I. (1995) Annu. Rev. Microbiol 49: 201-238
Katz L., Donadio S. (1993) Annu. Rev. Microbiol. 47: 875-912
Kuczek, K., Kotowska, M., Wiernik, D. & Mordarski, M. (1998) BioTechniques 24, 214-215.
Tang L., Fu H., McDaniel R. (2000) Chem. Biol. 7: 77-84
Xue Y, Wilson D., Sherman D. H. (2000) Gene 245: 203-211 Antibiot. 53: 484-495
Kieser T., Bibb M. J., Buttner M. J., Chater K. F., Hopwood D. A. (2000) Practical Streptomyces Genetics, The John Innes Foundation, Norwich
Ranganathan, A., Timoney, M., Bycroft, M., Cortés, J., Thomas, I. P., Wilkinson, B., Kellenberger, L., Hanefeld, U., Galloway, I. S., Staunton, J. & Leadlay, P. F. (1999) Chemistry & Biology 6, 731-741.
Sambrook J., Fritsch E. F., Maniatis T. (1989) Molecular Cloning: a Laboratory Manual, Cold Spring Harbor Laboratory Press
Schneider, A. & Marahiel, M. A. (1998) Arch. Microbiol., 169, 404-410.
Shaw-Reid, C. A., Kelleher, N. L., Losey, H. C., Gehring, A. M., Berg, Ch. & Walsh, Ch. T. (1999) Chemistry & Biology 6, 385-400.
Weissman, K. J., Cameron, J. S., Hanefeld, U., Aggarwal, R., Bycroft, M., Staunton, J. & Leadlay, P. F. (1998) Chem. Int. Ed., 37, 1437-1440.
Xue, Y., Wilson, D. & Sherman, D. H. (2000) Gene 245, 203-211.

1. An isolated polynucleotide comprising the sequence having at least 60% homology to the nucleic acid sequence of SEQ ID NO. 1 or its complementary strand or fragments thereof.

2. The polynucleotide of claim 1, comprising the sequence having at least 70% homology to the nucleic acid sequence of SEQ ID NO:1 or its complementary strand or fragments thereof.

3. The polynucleotide of claim 1, comprising the sequence having at least 80% homology to the nucleic acid sequence of SEQ ID NO:1 or its complementary strand or fragments thereof.

4. The polynucleotide of claim 1, comprising the sequence having at least 90% homology to the nucleic acid sequence of SEQ ID NO:1 or its complementary strand or fragments thereof.

5. The polynucleotide according to one of claim 1-4, characterised in that encodes tioesterase type II proteine.

6. The polynucleotide of claim 1, wherein said polynucleotide encodes at least 15 contiguous amino acids from SEQ ID NO:2.

7. The polynucleotide of claim 1, wherein said polynucleotide encodes at least 50 contiguous amino acids from SEQ ID NO:2.

8. The polynucleotide of claim 1, wherein said polynucleotide encodes at least 150 contiguous amino acids from SEQ ID NO:2.

9. The polynucleotide of claim 1, wherein said polynucleotide encodes polypeptide comprising the amino acid sequence of SEQ ID NO:2.

10. The polynucleotide of claim 1 or 9, comprising the sequence of SEQ ID NO:1 or SEQ ID NO:3 or complementary strand thereof.

11. A nucleic acid vector comprising the polynucleotide according to anyone of claims 1 to 9.

12. A protein or fragment thereof encoded by a polynucleotide according to anyone of claims 1 to 10.

13. A use of a polynucleotide according to anyone of claims 1 to 10 for an expression of a protein.

14. The use according to claim 13 wherein the protein have the TE II activity.

15. A use of a polypeptide comprising an amino acid sequence essentially consistent with the sequence SEQ ID No. 2 or fragments thereof for polyketide synthesis.

16. The use according to claim 15 characterized in, that the protein is used in a multienzyme polyketide synthase assembly.

17. The use according to claim 15 characterized in, that the protein is used in order to increase biosynthetic efficiency of a polyketide synthase complex.
